(19) United States
(12) Patent Application Publication
(10) Pub. No.: US 2003/0099601 A1
(43) Pub. Date: May 29, 2003

(54) Inhalation lung surfactant therapy

(76) Inventors: Marc S. Gordon, Sunnyvale, CA (US); Thomas Tarara, Burlingame, CA (US); Jeffry Weers, Half Moon Bay, CA (US)

Correspondence Address: NEKTAR THERAPEUTICS, 150 INDUSTRIAL ROAD, SAN CARLOS, CA, 94070, US

(21) Appl. No.: 10/302,553

(22) Filed: Nov. 22, 2002

Related U.S. Application Data
(60) Provisional application No. 60/333,729, filed on Nov. 27, 2001.

(51) Int. Cl. A61L 9/04; A61K 9/14

(52) U.S. Cl. 424/46

(57) ABSTRACT

Methods for providing lung surfactant therapy are provided. In particular, lung surfactant therapies effective to decrease oxygen index by inhalation of a dry powder aerosol composition are provided. The powders are preferably hollow and porous and administered via inhalation.

### FIELD OF THE INVENTION

The present invention relates to lung surfactant therapy by inhalation. In particular, the present invention provides lung surfactant particulate compositions and methods for their administration in order to provide effective lung surfactant therapy.

### BACKGROUND OF THE INVENTION

Phospholipids are major components of cell and organelle membranes, blood lipoproteins, and lung surfactant. In terms of pulmonary drug delivery, phospholipids have been investigated as therapeutic agents for the treatment of respiratory distress syndrome (i.e. exogenous lung surfactants), and as suitable excipients for the delivery of actives. The interaction of phospholipids with water is critical to the formation, maintenance, and function of each-of these important biological complexes (McIntosh and Magid). At low temperatures in the gel phase, the acyl chains are in a conformationally well-ordered state, essentially in the all-trans configuration. At higher temperatures, above the chain melting temperature, this chain order is lost, owing to an increase in gauche conformer content (Seddon and Cevc).

Several exogenous lung surfactants have been marketed and include products derived from bovine lungs (Survanta®, Abbott Laboratories), porcine lungs (CuroSurf®, Dey Laboratories), or completely synthetic surfactants with no apoproteins (e.g. ALEC®, ExoSurf® Glaxo Wellcome). To date, these products have been utilized for the treatment of infant respiratory distress syndrome (IRDS). None have been successful in receiving FDA approval for the treatment of adult respiratory distress syndrome (ARDS). The current infant dose is 100 mg/kg. For a 50 kg adult, this would translate into a dose of 5 g. A dose of this amount can only be administered to ARDS patients by direct instillation into the patient's endotracheal tube, or possibly via nebulization of aqueous dispersions of the surfactant material.

Instillation of surfactants leads to deposition primarily in the central airways, and little of the drug makes it to the alveoli, where it is needed to improve gas exchange in these critically ill patients. Nebulization of surfactant may allow for greater peripheral delivery, but is plagued by the fact that (a) current nebulizers are inefficient devices and only ca. 10% of the drug actually reaches the patients lungs; (b) the surfactant solutions foam during the nebulization process, leading to complications and further loss of drug. It is believed that as much as 99% of the administered surfactant may be wasted due to poor delivery to the patient. If more effective delivery of surfactant could be achieved, it is likely that the administered dose and cost for treatment of ARDS could be dramatically decreased.

Further, lung surfactant has been shown to modulate mucous transport in airways. In this regard, the chronic administration of surfactant for the treatment of patients with chronic obstructive pulmonary disease (COPD) has been suggested. Still other indications with significantly lower doses may be open to treatment if a dry powder form of a lung surfactant were available. The powdered surfactant formulation may be purely synthetic (i.e. with no added apoproteins). Alternatively, the powder formulation could contain the hydrophobic apoproteins SP—B or SP—C or alternative recombinant or synthetic peptide mimetics (e.g. KL₄).

Due to its spreading characteristics on lung epithelia, surfactant has been proposed as the ideal carrier for delivery of drugs to the lung, and via the lung to the systemic circulation. Once again, achieving efficient delivery to the lung is important, especially in light of the potential high cost of many of the current products. One potential way to deliver drugs in phospholipids is as a dry powder aerosolized to the lung. Most fine powders (<5 μm) exhibit poor dispersibility. This can be problematic when attempting to deliver, aerosolize, and/or package the powders.

Phospholipids are especially difficult to formulate as dry powders as their low gel to liquid crystal transition temperature (Tm) values and amorphous nature lead to powders which are very sticky and difficult to deaggregate and aerosolize. Phospholipids with Tm values less than 10° C. (e.g. egg PC or any unsaturated lipids) form highly cohesive powders following spray-drying. Inspection of the powders via scanning electron microscopy reveals highly agglomerated particles with surfaces that appear to have been melted/annealed. Formulating phospholipid powders which have low Tm are problematic, especially if one hopes to achieve a certain particle morphology, as in the case of aerosol delivery. Examples of particulate compositions incorporating a surfactant are disclosed in PCT publications WO 99/16419, WO 99/38493, WO 99/66903, WO 00/10541, and U.S. Pat. Nos. 5,855,913, which are hereby incorporated in their entirety by reference.

Currently, lung surfactant is given to patients by intubating them and instilling a suspension of lung surfactant directly into the lungs. This is a highly invasive procedure which generally is not performed on conscious patients, and as do most procedures, carries its own risks. Potential applications for lung surfactant beyond the current indication of respiratory distress syndrome in neonates are greatly limited by this method of administration. For example, lung surfactant may be useful in a variety of disease states that are, in part, due to decreased lung surfactant being present in the lungs. U.S. Pat. Nos. 5,451,569, 5,698,537, and 5,925,337, and PCT publications WO 97/26863 and WO 00/27360, for example, disclose the pulmonary administration of lung surfactant to treat various conditions, the disclosures of which are hereby incorporated in their entirety by reference. Diseases and procedures that are thought to be possibly aggravated by lung surfactant deficiency include cystic fibrosis, chronic obstructive pulmonary disease, bronchiectasis, cardiopulmonary bypass, interstitial lung diseases (including idiopathic pulmonary fibrosis), lung transplantation, pneumonia, thoracic irradiation injury, chronic asthma, and asthma, just to name a few. The delivery of exogenous lung surfactant, in a topical fashion, to patients suffering from these diseases may ameliorate certain signs and symptoms of the diseases. For chronic conditions, the regular (once or more times per day on a prolonged basis) delivery of lung surfactant via intubation and instillation to ambulatory patients is impractical. Further, because of their high surface activity, lung surfactant suspensions are not amenable to nebulization due to foaming. The current delivery of phospholipid -based preparations by instillation or nebulization are highly inefficient in delivering material to the peripheral lung. Therefore, the ability to deliver lung surfactant to patients via dry powder inhalation would be a tremendous advantage over the current method, since it would avoid the need for intubation, thereby expanding the potential uses of lung surfactant in the clinical setting.

### SUMMARY OF THE INVENTION

The present invention provides methods for effective surfactant therapy by inhalation. According to the invention, dry powder compositions of lung surfactant are efficiently delivered to the deep lung. The use of dry powder compositions may also open new indications for use since the patient need not be intubated. According to one embodiment, the compositions of the present invention may be delivered from a simple passive DPI device. The present compositions allow for greater stability on storage, and for more efficient delivery to the lung.

According to the invention, a method for providing lung surfactant therapy to a patient in need thereof is disclosed, which method comprises providing a dry powder composition comprising a lung surfactant and administering the composition by inhalation to the respiratory tract in order to produce a decrease in oxygen index of at least 20%, preferably at least 40%, and most preferably by at least 60%.

The composition preferably comprises calcium and is administered as a dry powder aerosol at a dose of at least 0.1 mg/kg. The composition is administered in a plurality of inhalations, such as up to 30 and preferably less than 10 over a 30 minute period or less. The method is effective to decrease the oxygen index for a period of at least 10 minutes, preferably at least 25 minutes, and most preferably at least 45 minutes.

According to a preferred aspect, the present invention provides a method for providing lung surfactant therapy to a patient in need thereof comprising providing a dry powder composition comprising a lung surfactant and administering said composition by inhalation to the respiratory tract in order to produce a decrease in oxygen index of at least 20%. The composition is preferably administered as a dry powder aerosol at an administered dose of at least 0.1 mg/kg.

It is a further aspect of the present invention to provide a powdered, dispersible form of a lung surfactant having stable dispersibility over time and excellent spreading characteristics on an aqueous subphase.

### Definitions

As used herein, the term “emitted dose” or “ED” refers to an indication of the delivery of dry powder from a suitable inhaler device after a firing or dispersion event from a powder unit or reservoir. ED is defined as the ratio of the dose delivered by an inhaler device (described in detail below) to the nominal dose (i.e., the mass of powder per unit dose placed into a suitable inhaler device prior to firing). The ED is an experimentally-determined amount, and is typically determined using an in-vitro device set up which mimics patient dosing. To determine an ED value, a nominal dose of dry powder (as defined above) is placed into a suitable dry powder inhaler, which is then actuated, dispersing the powder. The resulting aerosol cloud is then drawn by vacuum from the device, where it is captured on a tared filter attached to the device mouthpiece. The amount of powder that reaches the filter constitutes the delivered dose. For example, for a 5 mg, dry powder-containing blister pack placed into an inhalation device, if dispersion of the powder results in the recovery of 4 mg of powder on a tared filter as described above, then the ED for the dry powder composition is: 4 mg (delivered dose)/5 mg (nominal dose)×100=80%.

“Lung surfactant” as used herein refers to Infasurf® (Ony, Inc.) containing compositions which comprise an extract of natural surfactant from calf lungs which includes phospholipids such as phosphatidylcholine, neutral lipids, and hydrophobic surfactant-associate proteins B and C (SP—B and SP—C).

“Mass median diameter” or “MMD” is a measure of mean particle size, since the powders of the invention are generally polydisperse (i.e., consist of a range of particle sizes). MMD values as reported herein are determined by laser diffraction, although any number of commonly employed techniques can be used for measuring mean particle size.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

“Oxygen Index” is a derived value that attempts to quantitate severity of respiratory failure and is calculated by the equation Oxygen Index (OI)=% O₂×MAP/PₐO₂ where % O₂ is the percentage of inspired gas that is oxygen concentration as measured by an in-line oxygen analyzer; MAP is “mean airway pressure” measured in cm H₂O by electronically averaging the analog airway pressure over 5 seconds; and PₐO₂ is partial pressure of oxygen in arterial blood (torr or mmHg) and is measured directly using a blood gas analyzer. Severe RDS is characterized as having an OI>9.

### DETAILED DESCRIPTION OF THE INVENTION

The present invention provides a therapeutically effective lung surfactant therapy via pulmonary administration of a dry powder composition containing lung surfactant.

The lung surfactant compositions suitable for use in the present invention additionally include any of those phospholipids known in the art which is incorporated with the lung surfactant during processing according to the present invention. According to a preferred embodiment, such phospholipids are most preferably a saturated phospholipid. According to a particularly preferred embodiment, saturated phosphatidylcholines are used as the phospholipid of the present invention. Preferred acyl chain lengths are 16:0 and 18:0 (i.e. palmitoyl and stearoyl).

Phospholipids from both natural and synthetic sources are compatible with the present invention and may be used in varying concentrations to form the structural matrix. Generally compatible phospholipids comprise those that have a gel to liquid crystal phase transition greater than about 40° C. Preferably the incorporated phospholipids are relatively long chain (i.e. C₁₆-C₂₂) saturated lipids and more preferably comprise saturated phospholipids, most preferably saturated phosphatidylcholines having acyl chain lengths of 16:0 or 18:0 (palmitoyl and stearoyl). Exemplary phospholipids useful in the disclosed stabilized preparations comprise, 1-palmitoyl-2-oleoyl-SN-glycero-3-phosphoglycerol (POPG), phosphoglycerides such as dipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, long-chain saturated phosphatidylinositols.

In addition to the phospholipid, a co-surfactant or combinations of surfactants, including the use of one or more in the liquid phase and one or more associated with the particulate compositions are contemplated as being within the scope of the invention. By “associated with or comprise” it is meant that the particulate compositions may incorporate, adsorb, absorb, be coated with or be formed by the surfactant. Surfactants include fluorinated and nonfluorinated compounds and are selected from the group consisting of saturated and unsaturated lipids, nonionic detergents, nonionic block copolymers, ionic surfactants and combinations thereof. In those embodiments comprising stabilized dispersions, such nonfluorinated surfactants will preferably be relatively insoluble in the suspension medium. It should be emphasized that, in addition to the aforementioned surfactants, suitable fluorinated surfactants are compatible with the teachings herein and may be used to provide the desired preparations.

Compatible nonionic detergents suitable as co-surfactants comprise: sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.) which is incorporated herein in its entirety. Preferred block copolymers include diblock and triblock copolymers of polyoxyethylene and polyoxypropylene, including poloxamer 188 (Pluronic™ F-68), poloxamer 407 (Pluronic™ F-127), and poloxamer 338. Ionic surfactants such as sodium sulfosuccinate, and fatty acid soaps may also be utilized.

Other lipids including glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate may also be used in accordance with the teachings of this invention.

With respect to inhalation therapies, those skilled in the art will appreciate that the microparticles of the present invention are particularly useful in DPIs. Conventional DPIs comprise powdered formulations and devices where a predetermined dose of medicament, either alone or in a blend with lactose carrier particles, is delivered as an aerosol of dry powder for inhalation.

The medicament is formulated in a way such that it readily disperses into discrete particles with an MMD between 0.5 to 20 μm, preferably 0.5-5 μm, and are further characterized by an aerosol particle size distribution less than about 10 Jim mass median aerodynamic diameter (MMAD), and preferably less than 5.0 μm. The mass median aerodynamic diameters of the powders will characteristically range from about 0.5-10 μm, preferably from about 0.5-5.0 μm MMAD, more preferably from about 1.0-4.0 μm MMAD.

The powder is actuated either by inspiration or by some external delivery force, such as pressurized air. Examples of DPIs suitable for administration of the particulate compositions of the present invention are disclosed in U.S. Pat. Nos. 5,740,794, 5,785,049, 5,673,686, and 4,995,385 and PCT application nos. 00/72904, 00/21594, and 01/00263, hereby incorporated in their entirety by reference. DPI formulations are typically packaged in single dose units such as those disclosed in the above mentioned patents or they employ reservoir systems capable of metering multiple doses with manual transfer of the dose to the device.

Particularly preferred embodiments of the invention incorporate spray dried, hollow and porous particulate compositions as disclosed in WO 99/16419, hereby incorporated in its entirety by reference. Such particulate compositions comprise particles having a relatively thin porous wall defining a large internal void, although, other void containing or perforated structures are contemplated as well.

Compositions according to the present invention typically yield powders with bulk densities less than 0.5 g/cm³, preferably less than 0.2 g/cm³. By providing particles with very low bulk density, the minimum powder mass that can be filled into a unit dose container is reduced, which eliminates the need for carrier particles. That is, the relatively low density of the powders of the present invention provides for the reproducible administration of relatively low dose pharmaceutical compounds. Moreover, the elimination of carrier particles will potentially minimize throat deposition and any “gag” effect, since the large lactose particles will impact the throat and upper airways due to their size.

It will be appreciated that the particulate compositions disclosed herein comprise a structural matrix that exhibits, defines or comprises voids, pores, defects, hollows, spaces, interstitial spaces, apertures, perforations or holes. The absolute shape (as opposed to the morphology) of the perforated microstructure is generally not critical and any overall configuration that provides the desired characteristics is contemplated as being within the scope of the invention. Accordingly, preferred embodiments can comprise approximately microspherical shapes. However, collapsed, deformed or fractured particulates are also compatible.

In accordance with the teachings herein the particulate compositions will preferably be provided in a “dry” state. That is the microparticles will possess a moisture content that allows the powder to remain chemically and physically stable during storage at ambient temperature and easily dispersible. As such, the moisture content of the microparticles is typically less than 6% by weight, and preferably less 3% by weight. In some instances the moisture content will be as low as 1% by weight. Of course it will be appreciated that the moisture content is, at least in part, dictated by the formulation and is controlled by the process conditions employed, e.g., inlet temperature, feed concentration, pump rate, and blowing agent type, concentration and post drying.

Reduction in bound water leads to significant improvements in the dispersibility and flowability of phospholipid based powders, leading to the potential for highly efficient delivery of powdered lung surfactants. The improved dispersibility allows simple passive DPI devices to be used to effectively deliver these powders.

As seen from the passages above, various components may be associated with, or incorporated in the particulate compositions of the present invention. Similarly, several techniques may be used to provide particulates having the desired morphology (e.g. a perforated or hollow/porous configuration), dispersibility and density. Among other methods, particulate compositions compatible with the instant invention may be formed by techniques including spray drying, vacuum drying, solvent extraction, spray freeze drying, emulsification or lyophilization, and combinations thereof. It will further be appreciated that the basic concepts of many of these techniques are well known in the prior art and would not, in view of the teachings herein, require undue experimentation to adapt them so as to provide the desired particulate compositions.

While several procedures are generally compatible with the present invention, particularly preferred embodiments typically comprise particulate compositions formed by spray drying. As is well known, spray drying is a one-step process that converts a liquid feed to a dried particulate form. With respect to pharmaceutical applications, it will be appreciated that spray drying has been used to provide powdered material for various administrative routes including inhalation. See, for example, M. Sacchetti and M. M. Van Oort in: Inhalation Aerosols: Physical and Biological Basis for Therapy, A. J. Hickey, ed. Marcel Dekkar, New York, 1996, which is incorporated herein by reference.

Examples of spray drying methods and systems suitable for making the dry powders of the present invention are disclosed in WO 99/16419, copending U.S. application Ser. No. 09/851,226, U.S. Pat. Nos. 6,077,543, 6,051,256, 6,001,336, 5,985,248, and 5,976,574, hereby incorporated in their entirety by reference.

Although the particulate compositions are preferably formed using a blowing agent as described in detail in WO 99/16419 listed above, it will be appreciated that, in some instances, no additional blowing agent is required and an aqueous dispersion of the medicament and/or excipients and surfactant(s) are spray dried directly. In such cases, the formulation may be amenable to process conditions (e.g., elevated temperatures) that may lead to the formation of hollow, relatively porous microparticles. Moreover, the medicament may possess special physicochemical properties (e.g., high crystallinity, elevated melting temperature, surface activity, etc.) that makes it particularly suitable for use in such techniques.

In order to maximize dispersibility, dispersion stability and optimize distribution upon administration, the mean geometric particle size of the particulate compositions is preferably about 0.5-50 μm, more preferably 1-20 μm and most preferably 0.5-5 μm. It will be appreciated that large particles (i.e. greater than 50 μm) may not be preferred in applications where a valve or small orifice is employed, since large particles tend to aggregate or separate from a suspension which could potentially clog the device. In especially preferred embodiments the mean geometric particle size (or diameter) of the particulate compositions is less than 20 μm or less than 10 μm. More preferably the mean geometric diameter is less than about 5 μm. Other preferred embodiments will comprise preparations wherein the mean geometric diameter of the particulate compositions is between about 1 μm and 5 μm. In especially preferred embodiments the particulate compositions will comprise a powder of dry, hollow, porous microspherical particulates of approximately 1 to 5 μm in diameter, with shell thicknesses of approximately 0.1 μm to approximately 0.5 μm. It is a particular advantage of the present invention that the particulate concentration of the dispersions and structural matrix components can be adjusted to optimize the delivery characteristics of the selected particle size.

The foregoing description will be more fully understood with reference to the following Examples. Such Examples, are, however, merely representative of preferred methods of practicing the present invention and should not be read as limiting the scope of the invention.

### EXAMPLE I

The preparation of lung surfactant powders used in an in vivo study in lambs were prepared according to the following procedure:

The annex solution comprising 48 g water, 1.71 g Infasurf® (Ony, Inc.) material excluding water content, and 0.197 g calcium chloride dihydrate was prepared. The mixture was vortexed until all solids were fully dissolved.

Emulsions were prepared by adding 1.60 g DPPC into a beaker containing an amount (80.0 g) of 70° C. deionized water. The mixture was mixed in a Ultra Turrex mixer on low speed for approximately 1.5 minutes. 72.0 g PFOE was weighed out into a small flask and added dropwise into the DPPC/water mixture. The PFOE was added slowly over the course of 2-3 minutes and the mixture was then allowed to continue mixing for an additional 1-2 minutes.

The DPPC/water/PFOE mixture was then immediately removed from the mixer and run through an Emulsiflex C-5 homogenizer four times at 10,000-13,000 psi. The sample was then run through a homogenizer a fifth time at 13,000-17,000 psi. Two separate portions having a total mass of approximately 51 g were then weighed out.

The annex solution was then added to one of the DPPC/water/PFOE emulsion portions with continued stirring on a hot plate set to the lowest temperature. The mixtures were kept at approximately 50° C. with stirring during spray drying on a Bucchi 190 spray dryer under the following conditions:

| | |
|---|---|
| Atomization pressure: | 70 psi |
| Inlet temperature: | 99-106 C. |
| Outlet temperature: | 57-59 C. |
| Feed stock flow rate: | 5 ml/min |

### EXAMPLE II

In an in vitro test, the Infasurf powders prepared according to Example I adsorbed to an equilibrium surface tension of 23 mN/m within 30 seconds and reached a dynamic minimum surface tension of <1 mN/m on a pulsating bubble surfactometer (PBS Model No. EC-PBS-B, Electronetics Corporation, Buffalo, N.Y.).

The biological activity of the powder was investigated in ventilated preterm surfactant deficient lambs delivered by hysterotomy at 133±1 days gestation using arterial blood gasses as the main outcome measure. Delivered lambs were ventilated and managed based on arterial blood gases. Quantitation of severity of respiratory insufficiency used the Oxygen Index (OI) where [OI=% O₂×MAP/PₐO₂] and severe RDS is an OI≧9 (60% O₂×MAP 10/PₐO_{2<70}).

The lung surfactant composition was delivered as a dry powder, 2.2 mg dispersed into 20 mL/kg body wt of air (approximately 60 mL) and given as a single positive pressure inspiration repeated at 20 second intervals. Lung deposition was estimated as 33-67% of dose (deposition/dose 0.7-1.5 mg, 0.2-0.5 mg/kg). Pilot studies in eight lambs with severe RDS using 10 doses in each produced an acute improvement in OI, decreasing mean OI from 19±8.6 to 9.8±5.5 in 6 of 8. The positive effect lasted 15-60 minutes.

An additional 6 lambs received a 3 times higher dose (30 doses over 10 minutes). The OI decreased in all 6, mean 21.8±23.7 to 7.1±2.8. Thus, the magnitude of the OI effect was further increased with the larger dosage indicating a dose response. This study demonstrates that an inhaleable dry powder form of lung surfactant is biophysically active and is biologically effective.

We claim:

1. A method for providing lung surfactant therapy to a patient in need thereof comprising:
providing a dry powder composition comprising lung surfactant;
administering said composition by inhalation to the respiratory tract in order to produce a decrease in oxygen index of at least 20%.

2. The method of claim 1 wherein the composition further comprises a phospholipid selected from the group consisting of dipalmitoyphosphatidylcholine and distearoylphosphatidylcholine.

3. The method of claim 1 wherein the composition is administered as a dry powder aerosol.

4. The method of claim 3 wherein the administered dose is at least 0.1 mg/kg.

5. The method of claim 1 wherein the decrease in oxygen index is at least 40%.

6. The method of claim 1 wherein the decrease in oxygen index is at least 50%.

7. The method of claim 1 wherein the decrease in oxygen index is at least 60%.

8. The method of claim 3 wherein the composition is administered in a plurality of inhalations over a 30 minute period.

9. The method of claim 8 wherein the composition is administered in a plurality of inhalations over a 10 minute period.

10. The method of claim 8 wherein the plurality of inhalations is less than 30.

11. The method of claim 10 wherein the plurality if inhalations is less than 10.

12. The method of claim 1 wherein the administration of the composition is effective to decrease the oxygen index for a period of at least 10 minutes.

13. The method of claim 1 wherein the administration of the composition is effective to decrease the oxygen index for a period of at least 25 minutes.

14. The method of claim 1 wherein the administration of the composition is effective to decrease the oxygen index for a period of at least 45 minutes.

15. The method of claim 2 wherein the composition further comprises calcium.
